(19) United States
(12) Patent Application Publication
(10) Pub. No.: US 2012/0077818 A1
(43) Pub. Date: Mar. 29, 2012

(54) COMPOSITIONS COMPRISING TRANSNORSERTRALINE AND SEROTONIN RECEPTOR 1A AGONISTS/ANTAGONISTS AND USES THEREOF

(75) Inventors: Una Campbell, Marlborough, MA (US); Judith Dunn, Boston, MA (US)

(73) Assignee: Sunovion Pharmaceuticals Inc., Marlborough, MA (US)

(21) Appl. No.: 13/320,246

(22) PCT Filed: May 12, 2010
(86) PCT No.: PCT/US10/34473

§ 371 Date: Dec. 9, 2011

Related U.S. Application Data
(60) Provisional application No. 61/177,997, filed on May 13, 2009.

(51) Int. Cl. A61K 31/135; A61K 31/496; A61P 25/00; A61P 3/04; A61P 25/18; A61P 25/22; A61P 25/30; A61K 31/404; A61P 25/24

(52) U.S. Cl. 514/252.15; 514/647; 514/415; 514/253.01; 514/253.11; 514/252.19; 514/254.11

(57) ABSTRACT

Provided herein are methods of and compositions for, treating, preventing and managing various neurological disorders. The methods comprise administering a transnorsertraline in combination with a serotonin receptor IA agonist, antagonist or modulator.

This application claims priority to U.S. Provisional Application No. 61/177,997, filed May 13, 2009, the entirety of which is incorporated herein by reference.

### 1. FIELD

Provided herein are methods and compositions for treating, preventing and managing affective disorders and other various neurological disorders.

### 2. BACKGROUND

2.1 Transnorsertraline

(1S,4S)-cis-4-(3,4-Dichlorophenyl)-1,2-3,4-tetrahydro-1-naphthalenamine, also known as norsertraline, is a metabolite of sertraline, which is marketed in the United States under the trade name Zoloft®. Its trans-isomers (“transnorsertraline”), i.e., (1R,4S)-trans-4-(3,4-dichlorophenyl)-1,2-3,4-tetrahydro-1-naphthalenamine and (1S,4R)-trans-4-(3,4-dichlorophenyl)-1,2-3,4-tetrahydro-1-naphthalenamine, which were described in, for example, U.S. Pat. No. 7,087,785 B2 (“the '785 patent”; incorporated herein by reference in its entirety), have the following chemical structures, respectively:

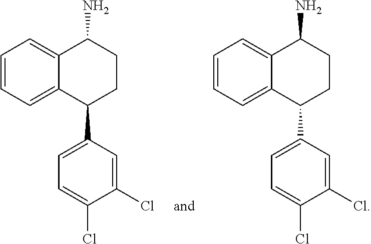

The primary clinical use of sertraline is in the treatment of depression. In addition, uses of transnorsertraline in the treatment, prevention, or management of affective disorders and other various CNS disorders are also disclosed in the '785 patent. Such disorders include, but are not limited to, depression, mood disorders, anxiety disorders, behavioral disorders, eating disorders, substance abuse disorders, and sexual function disorders.

2.2 Treatment of Neurological Disorders

Serotonin, i.e., 5-HT, is known to play an important role in the treatment of various CNS disorders. Among others, 5-HT1A (serotonin 1A) receptors provide an important mechanism for controlling 5-HT release in the brain. These receptors are located presynaptically in the raphe nuclei where they function as autoreceptors to inhibit the firing rate of 5-HT neurons. 5-HT_{1A} receptors are also located postsynaptically in corticolimbic regions where they also reduce firing activity of 5-HT neurons. At the initiation of treatment with selective serotonin reuptake inhibitors (SSRIs) or serotonin norepinephrine reuptake inhibitors (SNRIs), the 5-HT_{1A} autoreceptors are activated by 5-HT, leading to a reduction in 5-HT neuronal firing. As SSRI or SNRI treatment continues, however, 5-HT_{1A} autoreceptors become desensitized, and the firing activity is restored. This adaptive change is believed to contribute, at least in part, to the delay in efficacy of SSRIs and SNRIs in treating various neurological disorders.

Therefore, a need exists as to the treatment, prevention, or management of various neurological disorders, wherein the desensitization of 5-HT receptors may be minimized and the increase in 5-HT neuronal firing may be maintained.

### 3. SUMMARY

Provided herein are methods of treating, preventing or managing neurological disorders comprising administering to a subject (e.g., patient) a transnorsertraline, or a pharmaceutically acceptable salt or solvate (e.g., hydrate) thereof, in combination with a serotonin receptor 1A receptor agonist (full or partial), or a pharmaceutically acceptable salt, solvate, or stereoisomer thereof. Neurological disorders that may be treated, prevented, or managed by the methods provided herein are described in detail herein elsewhere.

Also provided are pharmaceutical compositions and dosage forms comprising a transnorsertraline, or a pharmaceutically acceptable salt or solvate (e.g., hydrate) thereof, and a serotonin receptor 1A receptor antagonist (full or partial), or a pharmaceutically acceptable salt, solvate, or stereoisomer thereof. The compositions and dosage forms may optionally comprise one or more other therapeutic agents.

Also provided are pharmaceutical compositions and dosage forms comprising a transnorsertraline, or a pharmaceutically acceptable salt or solvate (e.g., hydrate) thereof, and a modulator of 5-HT1A receptor, or a pharmaceutically acceptable salt, solvate, or stereoisomer thereof. The compositions and dosage forms may optionally comprise one or more other therapeutic agents.

### 4. BRIEF DESCRIPTION OF FIGURES

FIG. 1 illustrates the increase in dorsal raphe 5-HT firing rate obtained where (1R,4S)-trans-4-(3,4-dichlorophenyl)-1,2-3,4-tetrahydro-1-naphthalenamine was administered in combination with a serotonin 1A receptor antagonist WAY-100635.

FIG. 1A illustrates an example of integrated firing histograms showing the effects of intravenous doses of (1R,4S)-trans-4-(3,4-dichlorophenyl)-1,2-3,4-tetrahydro-1-naphthalenamine on the spontaneous activity of dorsal raphe 5-HT neurons in rats pretreated with WAY-100635.

FIG. 1B illustrate the mean(±SEM) of percent increase in basal firing rate observed at each dose of (1R,4S)-trans-4-(3,4-dichlorophenyl)-1,2-3,4-tetrahydro-1-naphthalenamine in dorsal raphe.

FIG. 1C illustrates the mean(±SEM) of the number of single spikes of 5-HT neurons.

FIG. 1D illustrates the mean(±SEM) of the number of bursts of 5-HT neurons.

### 5. DETAILED DESCRIPTION

Certain embodiments provided herein are based, in part, on a finding that a transnorsertraline, when used in combination with a serotonin 1A receptor agonist or antagonist, favorably affects the therapeutic efficacy for the treatment of neurological disorders. Without being limited by a particular theory, the combination of a transnorsertraline and a serotonin 1A receptor agonist or antagonist results in a significant enhancement in 5-HT neuron firing. Also without being limited by a particular theory, the combination of a transnorsertraline and a serotonin receptor 1A receptor agonist or antagonist may provide faster onset of anti-neurological disorder activity, improved efficacy for treating neurological disorders, and improved efficacy for treating treatment-resistant neurological disorders.

Accordingly, provided herein in certain embodiments are methods of and compositions for treating, preventing, and/or managing a central nervous system disorder.

5.1 Definition

As used herein, and unless otherwise indicated, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound provided herein, with or without other additional active agent, after the onset of symptoms of the particular disease.

As used herein, and unless otherwise indicated, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of disease or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing,” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a patient who had suffered from the particular disease in an attempt to prevent or minimize the recurrence of the disease.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice and the like. In specific embodiments, the subject is a human.

As used herein, and unless otherwise indicated, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like.

As used herein, and unless otherwise indicated, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein, and unless otherwise specified, the term “neurological disorder” refers to any condition of the central or peripheral nervous system of a mammal. The term “neurological disorder” includes, but is not limited to, neurodegenerative diseases (e.g., Alzheimer's disease, Parkinson's disease and amyotrophic lateral sclerosis), neuropsychiatric diseases (e.g., schizophrenia and anxieties, such as general anxiety disorder), and affective disorders (e.g., depression and attention deficit disorder). Exemplary neurological disorders include, but are not limited to, MLS (cerebellar ataxia), Huntington's disease, Down syndrome, multi-infarct dementia, status epilecticus, contusive injuries (e.g., spinal cord injury and head injury), viral infection induced neurodegeneration, (e.g., AIDS, encephalopathies), epilepsy, benign forgetfulness, closed head injury, sleep disorders, depression (e.g., bipolar disorder), dementias, movement disorders, psychoses, alcoholism, post-traumatic stress disorder and the like. “Neurological disorder” also includes any condition associated with the disorder. For instance, a method of treating a neurodegenerative disorder includes methods of treating loss of memory and/or loss of cognition associated with a neurodegenerative disorder. An exemplary method would also include treating or preventing loss of neuronal function characteristic of neurodegenerative disorder. “Neurological disorder” also includes any disease or condition that is implicated, at least in part, in monoamine (e.g., norepinephrine) signaling pathways (e.g., cardiovascular disease).

As used herein, and unless otherwise specified, the term “affective disorder” includes depression, attention deficit disorder, attention deficit disorder with hyperactivity, bipolar and manic conditions, and the like. The terms “attention deficit disorder” (ADD) and “attention deficit disorder with hyperactivity” (ADDH), or attention deficit/hyperactivity disorder (ADHD), are used herein in accordance with the accepted meanings as found in Diagnostic and Statistical Manual of Mental Disorders, 4^{th} Ed., American Psychiatric Association (1997) (DSM-IV™).

As used herein, and unless otherwise specified, the term “depression” includes all forms of depression including, but not limited to, major depressive disorder (MDD), bipolar disorder, seasonal affective disorder (SAD) and dysthymia. “Major depressive disorder” is used herein interchangeably with “unipolar depression” and “major depression.” “Depression” may also includes any condition commonly associated with depression, such as all forms of fatigue (e.g., chronic fatigue syndrome) and cognitive deficits.

As used herein, and unless otherwise specified, the terms “obsessive-compulsive disorder,” “substance abuse,” “pre-menstrual syndrome,” “anxiety,” “eating disorders” and “migraine” are used herein in a manner consistent with their accepted meanings in the art. See, e.g., DSM-IV™. For example, the term “eating disorder,” as used herein, refers to abnormal compulsions to avoid eating or uncontrollable impulses to consume abnormally large amounts of food. These disorders may affect not only the social well-being, but also the physical well-being of sufferers. Examples of eating disorders include, but are not limited to, anorexia nervosa, bulimia, and binge eating.

As used herein, and unless otherwise specified, the term “pain” refers to an unpleasant sensory and emotional experience. The term “pain,” as used herein, refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic peripheral neuropathic pain. See, e.g., Harrison's Principles of Internal Medicine, pp. 93-98 (Wilson et al., eds., 12th ed. 1991); Williams et al., J. Med. Chem. 42: 1481-1485 (1999), herein each incorporated by reference in their entirety. “Pain” is also meant to include mixed etiology pain, dual mechanism pain, allodynia, causalgia, central pain, hyperesthesia, hyperpathia, dysesthesia, and hyperalgesia. In addition, the term “pain” includes pain resulting from dysfunction of the nervous system: organic pain states that share clinical features of neuropathic pain and possible common pathophysiology mechanisms, but are not initiated by an identifiable lesion in any part of the nervous system.

The term “somatic pain,” as used herein, refers to a normal nerve response to a noxious stimulus such as injury or illness, e.g., trauma, burn, infection, inflammation, or disease process such as cancer, and includes both cutaneous pain (e.g., skin, muscle or joint derived) and visceral pain (e.g., organ derived).

The term “neuropathic pain,” as used herein, refers to a heterogeneous group of neurological conditions that result from damage to the nervous system. The term also refers to pain resulting from injury to or dysfunctions of peripheral and/or central sensory pathways, and from dysfunctions of the nervous system, where the pain often occurs or persists without an obvious noxious input. This includes pain related to peripheral neuropathies as well as central neuropathic pain. Common types of peripheral neuropathic pain include diabetic neuropathy (also called diabetic peripheral neuropathic pain, or DN, DPN, or DPNP), post-herpetic neuralgia (PHN), and trigeminal neuralgia (TGN). Central neuropathic pain, involving damage to the brain or spinal cord, can occur following stroke, spinal cord injury, and as a result of multiple sclerosis, and is also encompassed by the term. Other types of pain that are meant to be included in the definition of neuropathic pain include, but are not limited to, pain from neuropathic cancer pain, HIV/AIDS induced pain, phantom limb pain, and complex regional pain syndrome.

The term also encompasses the common clinical features of neuropathic pain including, but not limited to, sensory loss, allodynia (non-noxious stimuli produce pain), hyperalgesia and hyperpathia (delayed perception, summation, and painful aftersensation). Pain is often a combination of nociceptive and neuropathic types, for example, mechanical spinal pain and radiculopathy or myelopathy.

As used herein, and unless otherwise specified, the term “acute pain” refers to the normal, predicted physiological response to a noxious chemical, thermal or mechanical stimulus typically associated with invasive procedures, trauma and disease. It is generally time-limited, and may be viewed as an appropriate response to a stimulus that threatens and/or produces tissue injury. The term also refers to pain which is marked by short duration or sudden onset.

As used herein, and unless otherwise specified, the term “chronic pain” encompasses the pain occurring in a wide range of disorders, for example, trauma, malignancies and chronic inflammatory diseases such as rheumatoid arthritis. Chronic pain may last more than about six months. In addition, the intensity of chronic pain may be disproportionate to the intensity of the noxious stimulus or underlying process. The term also refers to pain associated with a chronic disorder, or pain that persists beyond resolution of an underlying disorder or healing of an injury, and that is often more intense than the underlying process would predict. It may be subject to frequent recurrence.

As used herein, and unless otherwise specified, the term “inflammatory pain” is pain in response to tissue injury and the resulting inflammatory process. Inflammatory pain is adaptive in that it elicits physiologic responses that promote healing. However, inflammation may also affect neuronal function. Inflammatory mediators, including PGE₂ induced by the COX2 enzyme, bradykinins, and other substances, bind to receptors on pain-transmitting neurons and alter their function, increasing their excitability and thus increasing pain sensation. Much chronic pain has an inflammatory component. The term also refers to pain which is produced as a symptom or a result of inflammation or an immune system disorder.

As used herein, and unless otherwise specified, the term “visceral pain” refers to pain which is located in an internal organ.

As used herein, and unless otherwise specified, the term “mixed etiology pain” refers to pain that contains both inflammatory and neuropathic components.

As used herein, and unless otherwise specified, the term “dual mechanism pain” refers to pain that is amplified and maintained by both peripheral and central sensitization.

As used herein, and unless otherwise specified, the term “causalgia” refers to a syndrome of sustained burning, allodynia, and hyperpathia after a traumatic nerve lesion, often combined with vasomotor and sudomotor dysfunction and later trophic changes. As used herein, and unless otherwise specified, the term “central pain” refers to pain initiated by a primary lesion or dysfunction in the central nervous system.

As used herein, and unless otherwise specified, the term “hyperesthesia” refers to increased sensitivity to stimulation, excluding the special senses.

As used herein, and unless otherwise specified, the term “hyperpathia” refers to a painful syndrome characterized by an abnormally painful reaction to a stimulus, especially a repetitive stimulus, as well as an increased threshold. It may occur with allodynia, hyperesthesia, hyperalgesia, or dysesthesia.

As used herein, and unless otherwise specified, the term “dysesthesia” refers to an unpleasant abnormal sensation, whether spontaneous or evoked. In certain embodiments, dysesthesia include hyperalgesia and allodynia.

As used herein, and unless otherwise specified, the term “hyperalgesia” refers to an increased response to a stimulus that is normally painful. It reflects increased pain on suprathreshold stimulation.

As used herein, and unless otherwise specified, the term “allodynia” refers to pain due to a stimulus that does not normally provoke pain.

As used herein, and unless otherwise specified, the term “Diabetic Peripheral Neuropathic Pain” (DPNP), also called diabetic neuropathy, DN or diabetic peripheral neuropathy), refers to chronic pain caused by neuropathy associated with diabetes mellitus. The classic presentation of DPNP is pain or tingling in the feet that can be described not only as “burning” or “shooting” but also as severe aching pain. Less commonly, patients may describe the pain as itching, tearing, or like a toothache. The pain may be accompanied by allodynia and hyperalgesia and an absence of symptoms, such as numbness.

As used herein, and unless otherwise specified, the term “Post-Herpetic Neuralgia”, also called “Postherpetic Neuralgia (PHN)”, refers to a painful condition affecting nerve fibers and skin. Without being limited by a particular theory, it is a complication of shingles, a second outbreak of the varicella zoster virus (VZV), which initially causes chickenpox.

As used herein, and unless otherwise specified, the term “neuropathic cancer pain” refers to peripheral neuropathic pain as a result of cancer, and can be caused directly by infiltration or compression of a nerve by a tumor, or indirectly by cancer treatments such as radiation therapy and chemotherapy (chemotherapy-induced neuropathy).

As used herein, and unless otherwise specified, the term “HIV/AIDS peripheral neuropathy” or “HIV/AIDS related neuropathy” refers to peripheral neuropathy caused by HIV/AIDS, such as acute or chronic inflammatory demyelinating neuropathy (AIDP and CIDP, respectively), as well as peripheral neuropathy resulting as a side effect of drugs used to treat HIV/AIDS.

As used herein, and unless otherwise specified, the term “Phantom Limb Pain” refers to pain appearing to come from where an amputated limb used to be. Phantom limb pain can also occur in limbs following paralysis (e.g., following spinal cord injury). “Phantom Limb Pain” is usually chronic in nature.

As used herein, and unless otherwise specified, the term “Trigeminal Neuralgia (TN)” refers to a disorder of the fifth cranial (trigeminal) nerve that causes episodes of intense, stabbing, electric-shock-like pain in the areas of the face where the branches of the nerve are distributed (lips, eyes, nose, scalp, forehead, upper jaw, and lower jaw). It is also known as the “suicide disease”.

As used herein, and unless otherwise specified, the term “Complex Regional Pain Syndrome (CRPS),” formerly known as Reflex Sympathetic Dystrophy (RSD), refers to a chronic pain condition whose key symptom is continuous, intense pain out of proportion to the severity of the injury, which gets worse rather than better over time. The term encompasses type 1 CRPS, which includes conditions caused by tissue injury other than peripheral nerve, and type 2 CRPS, in which the syndrome is provoked by major nerve injury, and is sometimes called causalgia.

As used herein, and unless otherwise specified, the term “fibromyalgia” refers to a chronic condition characterized by diffuse or specific muscle, joint, or bone pain, along with fatigue and a range of other symptoms. Previously, fibromyalgia was known by other names such as fibrositis, chronic muscle pain syndrome, psychogenic rheumatism and tension myalgias.

As used herein, and unless otherwise specified, the term “convulsion” refers to a neurological disorder and is used interchangeably with “seizure,” although there are many types of seizure, some of which have subtle or mild symptoms instead of convulsions. Seizures of all types may be caused by disorganized and sudden electrical activity in the brain. In some embodiments, convulsions are a rapid and uncontrollable shaking during which the muscles contract and relax repeatedly.

5.2 Methods of Treatment, Prevention and Management

In one embodiment, provided herein is a method of treating, preventing, or managing a central nervous system disorder comprising administering to a subject (e.g., patient) a therapeutically or prophylactically effective amount of a transnorsertraline, or a pharmaceutically acceptable salt or solvate thereof, and a full or partial serotonin 1A selective antagonist or agonist, or a pharmaceutically acceptable salt, solvate, or stereoisomer thereof.

In one embodiment, the transnorsertraline is (1R,4S)-transnorsertraline, i.e., (1R,4S)-trans-4-(3,4-dichlorophenyl)-1,2-3,4-tetrahydro-1-naphthalenamine. In another embodiment, the transnorsertraline is (1S,4R)-transnorsertraline, i.e., (1S,4R)-trans-4-(3,4-dichlorophenyl)-1,2-3,4-tetrahydro-1-naphthalenamine.

Examples of serotonin 1A selective antagonists or agonists that may be used in connection with the methods provided herein include, but are not limited to, buspirone (Buspar®), pindolol (Visken®), eltoprazine, tandospirone (Sediel®), lecozotan, AV-965, and WAY-100635.

In one embodiment, the serotonin 1A receptor agonist is buspirone (Buspar®). In another embodiment, the serotonin 1A receptor agonist is tandospirone (Sediel®). In another embodiment, the serotonin 1A receptor agonist is eltoprazine. In another embodiment, the serotonin 1A receptor antagonist is WAY-100635. In another embodiment, the serotonin 1A receptor antagonist is lecozotan. In another embodiment, the serotonin 1A receptor antagonist is AV-965. In another embodiment, the serotonin 1A receptor antagonist is pindolol (also described as a serotonin 1A partial agonist). In one embodiment, the transnorsertraline is (1S,4R)-transnorsertraline, and the serotonin 1A antagonist is WAY-100635. In another embodiment, the transnorsertraline is (1R,4S)-transnorsertraline, and the serotonin 1A antagonist is WAY-100635. WAY-100635 is described in, for example, Benjamin et al., Psychopharmacology, 188(2): 244-251 (2006), and is commercially available from, for example, Sigma/RBI (Oakville, ON, Canada).

In another embodiment, provided herein is a method of treating, preventing, or managing a central nervous system disorder comprising administering to a subject (e.g., patient) a therapeutically or prophylactically effective amount of a transnorsertraline, or a pharmaceutically acceptable salt or solvate thereof, and a serotonin 1A receptor modulator, or a pharmaceutically acceptable salt, solvate, or stereoisomer thereof.

In one embodiment, the transnorsertraline is (1S,4R)-transnorsertraline, and the serotonin receptor 1A antagonist is pindolol. In another embodiment, the transnorsertraline is (1R,4S)-transnorsertraline, and the serotonin receptor 1A antagonist is pindolol.

In one embodiment, the transnorsertraline is (1S,4R)-transnorsertraline, and the serotonin receptor 1A agonist is buspirone. In another embodiment, the transnorsertraline is (1R,4S)-transnorsertraline, and the serotonin receptor 1A agonist is buspirone. In one embodiment, the transnorsertraline is (1S,4R)-transnorsertraline, and the serotonin receptor 1A agonist is eltoprazine. In another embodiment, the transnorsertraline is (1R,4S)-transnorsertraline, and the serotonin receptor 1A agonist is eltoprazine.

In one embodiment, the transnorsertraline is (1S,4R)-transnorsertraline, and the serotonin receptor 1A agonist is tandospirone (Sediel®). In another embodiment, the transnorsertraline is (1R,4S)-transnorsertraline, and the serotonin receptor 1A agonist is tandospirone (Sediel®).

In one embodiment, the transnorsertraline is (1S,4R)-transnorsertraline, and the serotonin receptor 1A antagonist is lecozotan. In another embodiment, the transnorsertraline is (1R,4S)-transnorsertraline, and the serotonin receptor 1A antagonist is lecozotan.

In one embodiment, the transnorsertraline is (1S,4R)-transnorsertraline, and the serotonin receptor 1A antagonist is AV-965. In another embodiment, the transnorsertraline is (1R,4S)-transnorsertraline, and the serotonin receptor 1A antagonist is AV-965.

In some embodiment, the transnorsertraline may be used in combination with certain other agents that modulate the activity of serotonin at the serotonin 1A receptor. Such agents include serotonin 1A receptor modulators and partial serotonin 1A agonists or antagonists.

In some embodiments, the methods provided herein may optionally comprise the administration of one or more of other active agents. Such other agents include, but are not limited to, those drugs or therapies conventionally used for the treatment, prevention, and/or management of neurological disorders provided herein.

In one embodiment, provided herein is a method of effecting an anti-depressant-like effect. The method comprises administering to a subject (e.g., a mammal) a therapeutically effective amount of a transnorsertraline, or a pharmaceutically acceptable salt or solvate thereof, in combination with a selective serotonin 1A receptor agonist or antagonist. Anti-depressant-like effects may be measured using an animal model of disease, such as those known in the art and those described herein.

In other embodiments, the neurological disorder is: depression (e.g., major depressive disorder, bipolar disorder, unipolar disorder, dysthymia and seasonal affective disorder); cognitive deficits; fibromyalgia; pain (e.g., neuropathic pain); sleep related disorders (e.g., sleep apnea, insomnia, narcolepsy, cataplexy) including those sleep disorders which are produced by psychiatric conditions; chronic fatigue syndrome; attention deficit disorder (ADD); attention deficit hyperactivity disorder (ADHD); restless leg syndrome; schizophrenia; anxieties (e.g., general anxiety disorder, social anxiety disorder, panic disorder); obsessive compulsive disorder; posttraumatic stress disorder; seasonal affective disorder (SAD); premenstrual dysphoria; post-menopausal vasomotor symptoms (e.g., hot flashes, night sweats); neurodegenerative disease (e.g., Parkinson's disease, Alzheimer's disease and amyotrophic lateral sclerosis); manic conditions; dysthymic disorder; cyclothymic disorder; obesity; and substance abuse or dependency (e.g., cocaine addiction, nicotine addiction). In another embodiment, the compounds provided herein are useful to treat two or more conditions/disorders, which are comorbid, such as cognitive deficit and depression.

In certain embodiments, neurological disorders include cerebral function disorders, including without limitation, senile dementia, Alzheimer's type dementia, cognition, memory loss, amnesia/amnestic syndrome, epilepsy, disturbances of consciousness, coma, lowering of attention, speech disorders, Lennox syndrome, autism, and hyperkinetic syndrome.

Neuropathic pain includes without limitation post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use).

Other exemplary diseases and conditions that may be treated, prevented, and/or managed using the methods and/or compositions provided herein include, but are not limited to: obesity; migraine or migraine headache; urinary incontinence, including without limitation involuntary voiding of urine, dribbling or leakage of urine, stress urinary incontinence (SUI), urge incontinence, urinary exertional incontinence, reflex incontinence, passive incontinence, and overflow incontinence; and sexual dysfunction, in men or women, including without limitation sexual dysfunction caused by psychological and/or physiological factors, erectile dysfunction, premature ejaculation, vaginal dryness, lack of sexual excitement, inability to obtain orgasm, and psycho-sexual dysfunction, including without limitation, inhibited sexual desire, inhibited sexual excitement, inhibited female orgasm, inhibited male orgasm, functional dyspareunia, functional vaginismus, and atypical psychosexual dysfunction.

In one embodiment, the neurological disorder is depression. In another embodiment, the neurological disorder is anxiety disorder. In another embodiment, the neurological disorder is pain. In another embodiment, the neurological disorder is neuropathic pain. In another embodiment, the neuropathic pain is diabetic neuropathy.

In one embodiment, the neurological disorder is a neurodegenerative disease. In one embodiment, the neurodegenerative disease is Parkinson's disease. In another embodiment, the neurodegenerative disorder is Alzheimer's disease.

In one embodiment, the neurological disorder is incontinence, for example, urinary incontinence. In another embodiment, the neurological disorder is sexual dysfunction.

In one embodiment, the neurological disorder is obesity, and the therapeutically effective amount of compound to supply to a patient is sufficient so that said patient feels satiated.

In one embodiment, the compounds described herein treat, prevent, and/or manage a central nervous disorder, without causing addiction to said compounds.

Where a transnorsertraline is used in combination with a selective serotonin 1A receptor agonist or antagonist, the transnorsertraline and serotonin 1A receptor agonist or antagonist may be administered simultaneously or sequentially using the same or different routes of administration. In some embodiments, the transnorsertraline is administered prior to the administration of the serotonin 1A receptor agonist or antagonist. In other embodiments, the transnorsertraline and the serotonin 1A receptor agonist or antagonist are concurrently administered. In other embodiments, the transnorsertraline is administered after the administration of the serotonin 1A receptor agonist or antagonist.

Any suitable route of administration can be employed for providing the patient with a therapeutically or prophylactically effective dose of an active ingredient. For example, oral, mucosal (e.g., nasal, sublingual, buccal, rectal, vaginal), parenteral (e.g., intravenous, intramuscular), transdermal, and subcutaneous routes can be employed. Exemplary routes of administration include oral, transdermal, and mucosal. Suitable dosage forms for such routes include, but are not limited to, transdermal patches, ophthalmic solutions, sprays, and aerosols. Transdermal compositions can also take the form of creams, lotions, and/or emulsions, which can be included in an appropriate adhesive for application to the skin or can be included in a transdermal patch of the matrix or reservoir type as are conventional in the art for this purpose. An exemplary transdermal dosage form is a “reservoir type” or “matrix type” patch, which is applied to the skin and worn for a specific period of time to permit the penetration of a desired amount of active ingredient. The patch can be replaced with a fresh patch when necessary to provide constant administration of the active ingredient to the patient.

The amount to be administered to a subject (e.g., patient) to treat, prevent, and/or manage the disorders described herein will depend upon a variety of factors including the activity of the particular compound employed, the route of administration, the time of administration, the rate of excretion or metabolism of the particular compound being employed, the duration of the treatment, other drugs, compounds and/or materials used in combination with the particular compound employed, the age, sex, weight, condition, general health, and prior medical history of the patient being treated, and like factors well known in the medical arts.

A physician or veterinarian having ordinary skill in the art can readily determine and prescribe the effective amount required. For example, the physician or veterinarian could start doses of the compounds employed at levels lower than that required in order to achieve the desired therapeutic effect and gradually increase the dosage until the desired effect is achieved.

In general, a suitable daily dose of a compound provided herein will be that amount of the compound which is the lowest dose effective to produce a therapeutic or prophylactic effect. Such an effective dose will generally depend upon the factors described above. Generally, oral, intravenous, intracerebroventricular and subcutaneous doses of the compounds provided herein for a patient will range from about 0.005 mg per kilogram to about 5 mg per kilogram of body weight per day. In one embodiment, the oral dose of a compound provided herein will range from about 0.1 mg to about 5 g per day. In one embodiment, the oral dose of a compound provided herein will range from about 0.25 mg to about 2 g per day. In one embodiment, the oral dose of a compound provided herein will range from about 0.5 mg to about 1 g per day. In one embodiment, the oral dose of a compound provided herein will range from about 1 mg to about 500 mg per day. In another embodiment, the oral dose of a compound provided herein will range from about 2 mg to about 250 mg per day. In another embodiment, the oral dose of a compound provided herein will range from about 3 mg to about 300 mg per day. In one embodiment, the oral dose of a compound provided herein will range from about 5 mg to about 300 mg per day. In another embodiment, the oral dose of a compound provided herein will range from about 10 mg to about 100 mg per day. In another embodiment, the oral dose of a compound provided herein will range from about 25 mg to about 50 mg per day. In another embodiment, the oral dose of a compound provided herein will range from about 30 mg to about 200 mg per day. Each of the above-recited dosage ranges may be formulated as a single or multiple unit dosage formulations.

5.3 Pharmaceutical Compositions

In one embodiment, provided herein are pharmaceutical compositions comprising: a transnorsertraline, or a pharmaceutically acceptable salt or solvate thereof; a serotonin 1A receptor antagonist, or a pharmaceutically acceptable salt, solvate, or stereoisomer thereof; and a pharmaceutically acceptable carrier or excipient. Examples of suitable serotonin 1A receptor antagonists are provided herein elsewhere.

In another embodiment, provided herein are pharmaceutical compositions comprising: a transnorsertraline, or a pharmaceutically acceptable salt or solvate thereof; a serotonin 1A receptor agonist, or a pharmaceutically acceptable salt, solvate, or stereoisomer thereof; and a pharmaceutically acceptable carrier or excipient. Examples of suitable serotonin 1A receptor agonists are provided herein elsewhere.

In some embodiments, the pharmaceutical compositions provided herein may optionally comprise one or more other active agents. Examples of suitable agents are provided herein elsewhere.

Certain pharmaceutical compositions are single unit dosage forms suitable for oral, mucosal (e.g., nasal, sublingual, vaginal, buccal, trachea, bronchial, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic or hard gelatin capsules; cachets; troches; lozenges; dispersions; suppositories; ointments; cataplasms (poultices); pastes; powders; Unit Dose Vial (UDV) nebulized solutions; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

In one embodiment, the dosage form is an oral dosage form. In another embodiment, the oral dosage form is a capsule, tablet, or syrup. In another embodiment, the dosage form is a parenteral dosage form.

The formulation should suit the mode of administration. For example, oral administration may require enteric coatings to protect the compounds administered from degradation within the gastrointestinal tract. In another example, the compounds may be administered in a liposomal formulation to shield the compounds from degradative enzymes, facilitate transport in circulatory system, and effect delivery across cell membranes to intracellular sites.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms will vary from one another will be readily apparent to those skilled in the art. See, e.g., Remington's Pharmaceutical Sciences, 18th ed., Mack Publishing, Easton Pa. (1990).

The selected dosage level and frequency of administration of the pharmaceutical compositions provided herein will depend upon a variety of factors including the route of administration, the time of administration, the rate of excretion of the therapeutic agents, the duration of the treatment, other drugs, compounds and/or materials used in the patient, the age, sex, weight, condition, general health and prior medical history of the patient being treated, and like factors well known in the medical arts. For example, the dosage regimen is likely to vary with pregnant women, nursing mothers and children relative to healthy adults. A physician having ordinary skill in the art can readily determine and prescribe the therapeutically effective amount of the pharmaceutical composition required.

The pharmaceutical compositions provided herein may further comprise a pharmaceutically acceptable carrier. The term “pharmaceutically acceptable carrier” means one or more pharmaceutically acceptable excipients. Examples of such excipients are well known in the art and are listed in the USP (XXI)/NF (XVI), incorporated herein in its entirety by reference thereto, and include without limitation, binders, diluents, fillers, disintegrants, super disintegrants, lubricants, surfactants, antiadherents, stabilizers, and the like. The term “additives” is synonymous with the term “excipients,” as used herein.

The term “pharmaceutically acceptable” is used herein to refer to those compounds, materials, compositions and/or dosage forms which are, within the scope of sound medical judgment, suitable for administration to and for use in contact with the tissues and fluids of human beings and animals without excessive toxicity, irritation, allergic response, or other problem or complication, commensurate with a reasonable medically sound benefit/risk ratio.

Further, the term “pharmaceutically acceptable” excipient is employed to mean that there are no untoward chemical or physical incompatibilities between the active ingredients and any of the excipient components of a given dosage form. For example, an untoward chemical reaction is one wherein the potency of compounds used in methods and compositions provided herein is detrimentally reduced or increased due to the addition of one or more excipients. Another example of an untoward chemical reaction is one wherein the taste of the dosage form becomes excessively sweet, sour or the like to the extent that the dosage form becomes unpalatable. Each excipient must be “acceptable” in the sense of being compatible with the other ingredients of the formulation and not injurious to the patient.

Physical incompatibility refers to incompatibility among the various components of the dosage form and any excipient(s) thereof. For example, the combination of the excipient(s) and the active ingredient(s) may form an excessively hygroscopic mixture or an excessively segregated mixture to the degree that the desired shape of the dosage form (e.g., tablet, troche etc.), its stability or the like cannot be sufficiently maintained to be able to administer the dosage form in compliance with a prescribed dosage regimen as desired.

It is noted that all excipients used in the pharmaceutical compositions or dosage forms provided herein preferably meet or exceed the standards for pharmaceutical ingredients and combinations thereof in the USP/NF. The purpose of the USP/NF is to provide authoritative standards and specifications for materials and substances and their preparations that are used in the practice of the healing arts. The USP/NF establish titles, definitions, descriptions, and standards for identity, quality, strength, purity, packaging and labeling, and also, where practicable, provide bioavailability, stability, procedures for proper handling and storage and methods for their examination and formulas for their manufacture or preparation.

The stability of a pharmaceutical product may be defined as the capability of a particular formulation, in a specific container, to remain within its physical, chemical, microbiological, therapeutic and toxicological specification, although there are exceptions, and to maintain at least about 80%, preferably about 90%, more preferably about 95% of labeled potency level. Thus, for example, expiration dating is defined as the time in which the pharmaceutical product will remain stable when stored under recommended conditions.

Many factors affect the stability of a pharmaceutical product, including the stability of the therapeutic ingredient(s), the potential interaction between therapeutic and inactive ingredients and the like. Physical factors such as heat, light and moisture may initiate or accelerate chemical reactions.

5.3.1 Oral Dosage Forms

Pharmaceutical compositions provided herein that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington: The Science and Practice of Pharmacy, 20^{th} Ed. (2000).

Typical oral dosage forms are prepared by combining the active ingredients in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Large-scale production of pharmaceutical compositions or dosage forms in accordance with the present disclosure may require, in addition to the therapeutic drug ingredients, excipients or additives including, but not limited to, diluents, binders, lubricants, disintegrants, colorants, flavors, sweetening agents and the like or mixtures thereof. By the incorporation of these and other additives, a variety of dosage forms (e.g., tablets, capsules, caplets, troches and the like) may be made. These include, for example, hard gelatin capsules, caplets, sugar-coated tablets, enteric-coated tablets to delay action, multiple compressed tablets, prolonged-action tablets, tablets for solution, effervescent tablets, buccal and sublingual tablets, troches and the like.

Hence, unit dose forms or dosage formulations of a pharmaceutical composition provided herein, such as a troche, a tablet or a capsule, may be formed by combining a desired amount of each of the active ingredients with one or more pharmaceutically compatible or acceptable excipients, as described below, in pharmaceutically compatible amounts to yield a unit dose dosage formulation the desired amount of each active ingredient. The dose form or dosage formulation may be formed by methods well known in the art.

Tablets are often a preferred dosage form because of the advantages afforded both to the patient (e.g., accuracy of dosage, compactness, portability, blandness of taste as well as ease of administration) and to the manufacturer (e.g., simplicity and economy of preparation, stability as well as convenience in packaging, shipping and dispensing). Tablets are solid pharmaceutical dosage forms containing therapeutic drug substances with or without suitable additives.

Tablets are typically made by molding, by compression or by generally accepted tablet forming methods. Accordingly, compressed tablets are usually prepared by large-scale production methods while molded tablets often involve small-scale operations. For example, there are three general methods of tablet preparation: (1) the wet-granulation method; (2) the dry-granulation method; and (3) direct compression. These methods are well known to those skilled in the art. See, Remington: The Science and Practice of Pharmacy, 20^{th} Ed. (2000). See, also, U.S. Pharmacopeia XXI, U.S. Pharmacopeial Convention, Inc., Rockville, Md. (1985).

Various tablet formulations may be made in accordance with the methods and compositions provided herein. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be colored and are beneficial in covering up drug substances possessing objectionable tastes or odors and in protecting materials sensitive to oxidation. Film-coated tablets (FCT) are compressed tablets that are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in methods and compositions provided herein. Enteric-coated tablets (ECT) are compressed tablets coated with substances that resist dissolution in gastric fluid but disintegrate in the intestine. Enteric coating can be used for tablets containing drug substances that are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press-coated tablets) may be used, for example, in the design of prolonged-action dosage forms.

Pharmaceutical compositions or unit dosage forms provided herein in the form of prolonged-action tablets may comprise compressed tablets formulated to release the drug substance in a manner to provide medication over a period of time. There are a number of tablet types that include delayed-action tablets in which the release of the drug substance is prevented for an interval of time after administration or until certain physiological conditions exist. Repeat action tablets may be formed that periodically release a complete dose of the drug substance to the gastrointestinal fluids. Also, extended release tablets that continuously release increments of the contained drug substance to the gastrointestinal fluids may be formed.

In order for medicinal substances or therapeutic ingredients provided herein, with or without excipients, to be made into solid dosage forms (e.g., tablets) with pressure, using available equipment, it is necessary that the material, either in crystalline or powdered form, possess a number of physical characteristics. These characteristics can include, for example, the ability to flow freely, as a powder to cohere upon compaction, and to be easily released from tooling. Since most materials have none or only some of these properties, methods of tablet formulation and preparation have been developed to impart these desirable characteristics to the material which is to be compressed into a tablet or similar dosage form.

As noted, in addition to the drugs or therapeutic ingredients, tablets and similar dosage forms may contain a number of materials referred to as excipients or additives. These additives are classified according to the role they play in the formulation of the dosage form such as a tablet, a caplet, a capsule, a troche or the like. One group of additives include, but are not limited to, binders, diluents (fillers), disintegrants, lubricants, and surfactants. In one embodiment the diluent, binder, disintegrant, and lubricant are not the same.

A binder is used to provide a free-flowing powder from the mix of tablet ingredients so that the material will flow when used on a tablet machine. The binder also provides a cohesiveness to the tablet. Too little binder will give flow problems and yield tablets that do not maintain their integrity, while too much can adversely affect the release (dissolution rate) of the drugs or active ingredients from the tablet. Thus, a sufficient amount of binder should be incorporated into the tablet to provide a free-flowing mix of the tablet ingredients without adversely affecting the dissolution rate of the drug ingredients from the tablet. With lower dose tablets, the need for good compressibility can be eliminated to a certain extent by the use of suitable diluting excipients called compression aids. The amount of binder used varies upon the type of formulation and mode of administration, and is readily discernible to those of ordinary skill in the art.

Binders suitable for use with dosage formulations provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (povidone), methyl cellulose, pre-gelatinized starch, hydroxypropyl methyl cellulose, (e.g., Nos. 2208, 2906, 2910), microcrystalline cellulose or mixtures thereof. Suitable forms of microcrystalline cellulose can include, for example, the materials sold as AVICEL-PH-101, AVICEL-PH-103 and AVICEL-PH-105 (available from FMC Corporation, American Viscose Division, Avicel Sales, Marcus Hook, Pa., U.S.A.).

Fillers or diluents are used to give the powder (e.g., in the tablet or capsule) bulk so that an acceptable size tablet, capsule or other desirable dosage form is produced. Typically, therapeutic ingredients are formed in a convenient dosage form of suitable size by the incorporation of a diluent therewith. As with the binder, binding of the drug(s) to the filler may occur and affect bioavailability. Consequently, a sufficient amount of filler should be used to achieve a desired dilution ratio without detrimentally affecting release of the drug ingredients from the dosage form containing the filler. Further, a filler that is physically and chemically compatible with the therapeutic ingredient(s) of the dosage form should be used. The amount of filler used varies upon the type of formulation and mode of administration, and is readily discernible to those of ordinary skill in the art. Examples of fillers include, but are not limited to, lactose, glucose, sucrose, fructose, talc, calcium carbonate (e.g., granules or powder), microcrystalline cellulose, powdered cellulose, dextrates, kaolin, mannitol, silicic acid, sorbitol, starch, pre-gelatinized starch, or mixtures thereof.

Disintegrants are used to cause the dose form (e.g., tablet) to disintegrate when exposed to an aqueous environment. Too much of a disintegrant will produce tablets which may disintegrate in the bottle due to atmospheric moisture. Too little may be insufficient for disintegration to occur and may thus alter the rate and extent of release of drug(s) or active ingredient(s) from the dosage form. Thus, a sufficient amount of disintegrant that is neither too little nor too much to detrimentally alter the release of the drug ingredients should be used to form the dosage forms provided herein. The amount of disintegrant used varies based upon the type of formulation and mode of administration, and is readily discernible to the skilled artisan. Examples of disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof.

When a dose form that dissolves fairly rapidly upon administration to the subject, e.g., in the subject's stomach is desired, a super disintegrant can be used, such as, but not limited to, croscarmellose sodium or sodium starch glycolate. The term “super disintegrant,” as used herein, means a disintegrant that results in rapid disintegration of drug or active ingredient in the stomach after oral administration. Use of a super disintegrant can facilitate the rapid absorption of drug or active ingredient(s) which may result in a more rapid onset of action.

Adhesion of the dosage form ingredients to the punches of the manufacturing machine (e.g., a tableting machine) must be avoided. For example, when drug accumulates on the punch surfaces, it causes the tablet surface to become pitted and therefore unacceptable. Also, sticking of drug or excipients in this way requires unnecessarily high ejection forces when removing the tablet from the die. Excessive ejection forces may lead to a high breakage rate and increase the cost of production not to mention excessive wear and tear on the dies. In practice, it is possible to reduce sticking by wet-massing or by the use of lubricants, e.g., magnesium stearate. However, selection of a drug salt with good anti-adhesion properties can also minimize these problems.

As noted, the lubricant is used to enhance the flow of the tableting powder mix to the tablet machine and to prevent sticking of the tablet in the die after the tablet is compressed. Too little lubricant will not permit satisfactory tablets to be made and too much may produce a tablet with a water-impervious hydrophobic coating, which can form because lubricants are usually hydrophobic materials such as stearic acid, magnesium stearate, calcium stearate and the like. Further, a water-impervious hydrophobic coating can inhibit disintegration of the tablet and dissolution of the drug ingredient(s). Thus, a sufficient amount of lubricant should be used that readily allows release of the compressed tablet from the die without forming a water-impervious hydrophobic coating that detrimentally interferes with the desired disintegration and/or dissolution of the drug ingredient(s).

Example of suitable lubricants for use with the compositions provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, or mixtures thereof. Additional lubricants include, for example, a syloid silica gel (AEROSIL 200, manufactured by W. R. Grace Co. of Baltimore Md.), a coagulated aerosol of synthetic silica (marketed by Deaussa Co. of Plano, Tex.), CAB-O-SIL (a pyrogenic silicon dioxide product sold by Cabot Co. of Boston, Mass.) or mixtures thereof.

Surfactants are used in dosage forms to improve the wetting characteristics and/or to enhance dissolution, and are particularly useful in pharmaceutical compositions or dosage forms containing poorly soluble or insoluble drug(s) or active ingredients. Examples of surfactants include, but are not limited to, polyoxyethylene sorbitan fatty acid esters, such as those commercially available as TWEENs (e.g. Tween 20 and Tween 80), polyethylene glycols, polyoxyethylene stearates, polyvinyl alcohol, polyvinylpyrrolidone, poly(oxyethylene)/poly(oxypropylene) block co-polyers such as poloxamers (e.g., commercially available as PLURONICs), and tetrafunctional block copolymers derived from sequential addition of propylene oxide and ethylene oxide to ethylenediamine, such as polyxamines (e.g., commercially as TETRONICs (BASF)), dextran, lecithin, dialkylesters of sodium sulfosuccinic acid, such as Aerosol OT, sodium lauryl sulfate, alkyl aryl polyether sulfonates or alcohols, such as TRITON X-200 or tyloxapol, p-isononylphenoxypoly (glycidol) (e.g. Olin-10G or Surfactant 10-G (Olin Chemicals), or mixtures thereof. Other pharmaceutically acceptable surfactants are well known in the art, and are described in detail in the Handbook of Pharmaceutical Excipients.

Other classes of additives for use with the pharmaceutical compositions or dosage forms provided herein include, but are not limited to, anti-caking or antiadherent agents, antimicrobial preservatives, coating agents, colorants, desiccants, flavors and perfumes, plasticizers, viscosity increasing agents, sweeteners, buffering agents, humectants and the like.

Examples of anti-caking agents include, but are not limited to, calcium silicate, magnesium silicate, silicon dioxide, colloidal silicon dioxide, talc, or mixtures thereof.

Examples of antimicrobial preservatives include, but are not limited to, benzalkonium chloride solution, benzethonium chloride, benzoic acid, benzyl alcohol, butyl paraben, cetylpyridinium chloride, chlorobutanol, cresol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymol, or mixtures thereof.

Examples of colorants for use with compositions provided herein include, but are not limited to, pharmaceutically acceptable dyes and lakes, caramel, red ferric oxide, yellow ferric oxide or mixtures thereof. Examples of desiccants include, but are not limited to, calcium chloride, calcium sulfate, silica gel or mixtures thereof.

Flavors that may be used include, but are not limited to, acacia, tragacanth, almond oil, anethole, anise oil, benzaldehyde, caraway, caraway oil, cardamom oil, cardamom seed, compound cardamom tincture, cherry juice, cinnamon, cinnamon oil, clove oil, cocoa, coriander oil, eriodictyon, eriodictyon fluidextract, ethyl acetate, ethyl vanillin, eucalyptus oil, fennel oil, glycyrrhiza, pure glycyrrhiza extract, glycyrrhiza fluidextract, lavender oil, lemon oil, menthol, methyl salicylate, monosodium glutamate, nutmeg oil, orange flower oil, orange flower water, orange oil, sweet orange peel tincture, compound orange spirit, peppermint, peppermint oil, peppermint spirit, pine needle oil, rose oil, stronger rose water, spearmint, spearmint oil, thymol, tolu balsam tincture, vanilla, vanilla tincture, and vanillin or mixture thereof.

Examples of sweetening agents include, but are not limited to, aspartame, dextrates, mannitol, saccharin, saccharin calcium, saccharin sodium, sorbitol, sorbitol solution, or mixtures thereof.

Exemplary plasticizers for use with the compositions provided herein include, but are not limited to, castor oil, diacetylated monoglycerides, diethyl phthalate, glycerin, mono-and di-acetylated monoglycerides, polyethylene glycol, propylene glycol, and triacetin or mixtures thereof. Suitable viscosity increasing agents include, but are not limited to, acacia, agar, alamic acid, aluminum monostearate, bentonite, bentonite magma, carbomer 934, carboxymethylcellulose calcium, carboxymethylcellulose sodium, carboxymethylcellulose sodium 12, carrageenan, cellulose, microcrystalline cellulose, gelatin, guar gum, hydroxyethyl cellulose, hydroxypropyl cellulose, hydroxypropyl methylcellulose (Nos. 2208; 2906; 2910), magnesium aluminum silicate, methylcellulose, pectin, polyvinyl alcohol, povidone, silica gel, colloidal silicon dioxide, sodium alginate, tragacanth and xanthan gum or mixtures thereof.

Buffering agents that may be used in the compositions provided herein include, but are not limited to, magnesium hydroxide, aluminum hydroxide and the like, or mixtures thereof. Examples of humectants include, but are not limited to, glycerol, other humectants or mixtures thereof.

The dosage forms provided herein may further include one or more of the following: (1) dissolution retarding agents, such as paraffin; (2) absorption accelerators, such as quaternary ammonium compounds; (3) wetting agents, such as, for example, cetyl alcohol and glycerol monostearate; (4) absorbents, such as kaolin and bentonite clay; (5) antioxidants, such as water soluble antioxidants (e.g., ascorbic acid, cysteine hydrochloride, sodium bisulfate, sodium metabisulfate, sodium sulfite and the like), oil soluble antioxidants (e.g., ascorbyl palmitate, hydroxyanisole (BHA), butylated hydroxy toluene (BHT), lecithin, propyl gallate, alpha-tocopherol and the like); and (6) metal chelating agents, such as citric acid, ethylenediamine tetracetic acid (EDTA), sorbitol, tartaric acid, phosphoric acid and the like.

Dosage forms provided herein, such as a tablet or caplet, may optionally be coated. Inert coating agents typically comprise an inert film-forming agent dispersed in a suitable solvent, and may further comprise other pharmaceutically acceptable adjuvants, such as colorants and plasticizers. Suitable inert coating agents, and methods for coating, are well known in the art, including without limitation aqueous or non-aqueous film coating techniques or microencapsulation. Examples of film-forming or coating agents include, but are not limited to, gelatin, pharmaceutical glaze, shellac, sucrose, titanium dioxide, carnauba wax, microcrystalline wax, celluloses, such as methylcellulose, hydroxymethyl cellulose, carboxymethylcellulose, cellulose acetate phthalate, hydroxypropyl methylcellulose (e.g., Nos.: 2208, 2906, 2910), hydroxypropyl cellulose, hydroxypropyl methyl cellulose phthalate (e.g., Nos.: 200731, 220824), hydroxyethylcellulose, methylhydroxyethylcellulose, ethylcellulose which may optionally be cross-linked, and sodium carboxymethyl cellulose; vinyls, such as polyvinyl pyrrolidione, polyvinyl acetate phthalate; glycols, such as polyethylene glycols; acrylics, such as dimethylaminoethyl methacrylate-methacrylate acid ester copolymer, and ethylacrylate-methylmethacrylate copolymer; and other carbohydrate polymers, such as maltodextrins, and polydextrose, or mixtures thereof. The amount of coating agent and the carrier vehicle (aqueous or non-aqueous) used varies upon the type of formulation and mode of administration, and is readily discernible to those of ordinary skill in the art.

A coating of a film forming polymer may optionally be applied to a tablet or caplet (e.g., a capsule shaped tablet) by using one of several types of equipment such as a conventional coating pan, Accelacota, High-Cola or Worster air suspension column. Such equipment typically has an exhaust-system to remove dust and solvent or water vapors to facilitate quick drying. Spray guns or other suitable atomizing equipment may be introduced into the coating pans to provide spray patterns conducive to rapid and uniform coverage of the tablet bed. Normally, heated or cold drying air is introduced over the tablet bed in a continuous or alternate fashion with a spray cycle to expedite drying of the film coating solution.

The coating solution may be sprayed by using positive pneumatic displacement or peristaltic pump systems in a continuous or intermittent spray-dry cycle. The particular type of spray application is selected depending upon the drying efficiency of the coating pan. In most cases, the coating material is sprayed until the tablets are uniformly coated to the desired thickness and the desired appearance of the tablet is achieved. Many different types of coatings may be applied such as enteric, slow release coatings or rapidly dissolving type coatings for fast acting tablets. Preferably, rapidly dissolving type coatings are used to permit more rapid release of the active ingredients, resulting in hastened onset. The thickness of the coating of the film forming polymer applied to a tablet, for example, may vary. However, it is preferred that the thickness simulate the appearance, feel (tactile and mouth feel) and function of a gelatin capsule. Where more rapid or delayed release of the therapeutic agent(s) is desired, one skilled in the art would easily recognize the film type and thickness, if any, to use based on characteristics such as desired blood levels of active ingredient, rate of release, solubility of active ingredient, and desired performance of the dosage form.

A number of suitable film forming agents for use in coating a final dosage form, such as tablets include, for example, methylcellulose, hydroxypropyl methyl cellulose (PHARMACOAT 606 6 cps), polyvinylpyrrolidone (povidone), ethylcellulose (ETHOCEL 10 cps), various derivatives of methacrylic acids and methacrylic acid esters, cellulose acetate phthalate or mixtures thereof.

The method of preparation and the excipients or additives to be incorporated into dosage form (such as a tablet or caplet) are selected in order to give the tablet formulation the desirable physical characteristics while allowing for ease of manufacture (e.g., the rapid compression of tablets). After manufacture, the dose form preferably should have a number of additional attributes, for example, for tablets, such attributes include appearance, hardness, disintegration ability and uniformity, which are influenced both by the method of preparation and by the additives present in the tablet formulation.

Further, it is noted that tablets or other dosage forms of the pharmaceutical compositions provided herein should retain their original size, shape, weight and color under normal handling and storage conditions throughout their shelf life. Thus, for example, excessive powder or solid particles at the bottom of the container, cracks or chips on the face of a tablet, or appearance of crystals on the surface of tablets or on container walls are indicative of physical instability of uncoated tablets. Hence, the effect of mild, uniform and reproducible shaking and tumbling of tablets should be undertaken to insure that the tablets have sufficient physical stability. Tablet hardness can be determined by commercially available hardness testers. In addition, the in vitro availability of the active ingredients should not change appreciably with time.

The tablets, and other dosage forms of the pharmaceutical compositions provided herein, such as dragees, capsules, pills and granules, may optionally be scored or prepared with coatings and shells, such as enteric coatings and other coatings well known in the pharmaceutical formulating art.

5.3.2 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms provided herein are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients (i.e., the compounds used in methods and compositions provided herein) disclosed herein can also be incorporated into the parenteral dosage forms.

5.2.3 Transdermal, Topical and Mucosal Dosage Forms

Transdermal, topical, and mucosal dosage forms provided herein include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms provided herein are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients provided herein. For example, penetration enhancers can be used to assist in delivering the active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts or solvates (e.g., hydrates) of the active ingredients can be used to further adjust the properties of the resulting composition.

5.2.4 Compositions with Enhanced Stability

The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions provided herein can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

Further provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Also provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

5.3.5 Delayed Release Dosage Forms

Active ingredients used in methods and compositions provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the compounds used in methods and compositions provided herein. Thus, provided herein are single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.3.6 Kits

In some cases, active ingredients used in methods and compositions provided herein are preferably not administered to a patient at the same time or by the same route of administration. Therefore, provided are kits which, when used by the medical practitioner, can simplify the administration of appropriate amounts of active ingredients to a patient.

In one embodiment, the kit comprises a single unit dosage form of the compounds used in methods and composition provided herein, or a pharmaceutically acceptable salt, solvate, or stereoisomer thereof, and a single unit dosage form of another agent that may be used in combination with those compounds. Kits provided herein can further comprise devices that are used to administer the active ingredients. Examples of such devices include, but are not limited to, syringes, drip bags, patches, and inhalers.

Kits provided herein can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Certain embodiments are exemplified in the following non-limiting examples. It will be apparent to those skilled in the art that many modifications, both to materials and methods, can be practiced without departing from the spirit and scope of this disclosure.

### 6. EXAMPLES

6.1 Excitatory Effects of Transnorsertraline on 5-HT Neuronal Activity in Presence of a Selective Serotonin 1A Receptor Antagonist

6.1.1. Procedures

6.1.1.1 Animals

Male Sprague Dawley rats (Charles River, St. Constant, QC) weighing 250 to 300 g, were used for the experiments. They were housed individually and kept under standard laboratory conditions (12:12 hour light/dark cycle with free access to food and water). All animals were handled according to the guidelines of the Canadian Council on Animal Care (CCAC) and protocols in this study were approved by the local Animal Care Committee (Ottawa Health Research Institute, Ottawa, ON, Canada).

6.1.1.2 In Vivo Electrophysiological Recordings

Rats were anaesthetized with chloral hydrate (400 mg/kg; i.p.) and placed into a stereotaxic frame. The extracellular recordings of 5-HT neurons in the dorsal raphe (“DR”) were carried out using single-barrelled glass micropipettes (R&D Scientific Glass, Spencerville, Md.) preloaded with a 2 M NaCl solution. Their impedance typically ranged between 4-7 MΩ.

6.1.1.3 Recording of DR 5-HT Neurons

The single-barrelled glass micropipettes were positioned using the following coordinates (in mm from lambda): AP, +1.0 to 1.2; L, 0±0.1; V, 5 to 7. The presumed 5-HT neurons were then identified using the following criteria: a slow (0.5-2.5 Hz) and regular firing rate and long-duration (2-5 ms) bi- or triphasic extracellular waveform (Aghajanian and Vandermaelen, 1982b). As previously demonstrated, 5-HT neurons display a bursting activity (Hajos et al., Neuroscience, 69(1): 189-197 (1995)). This occasional firing pattern of 5-HT neurons was analyzed by spike interval burst analysis following the criteria set by Hajos et al. The onset of a burst was defined as the occurrence of two spikes with an interspike interval shorter than 0.005 s.

6.1.1.4 Assessment of Neuronal Responsiveness

The percent of baseline firing rate were measured 60 seconds after systemic administration of the triple reuptake inhibitor. Various parameters were determined to examine the electrophysiological effects of co-administration of a transnorsertraline with WAY-100635. These parameters included the number of single spikes, bursts, cells per track and the firing rate of 5-HT neurons.

6.1.1.5 Statistical Analysis

Electrophysiological data expressed as means±S.E.M of percent of baseline firing rate or as means±S.E.M of single spikes or bursts measured from the same neurons. Data were analyzed by using a one- or two-ways analysis of variance (ANOVA), with treatment, treatment and pre-treatment or treatment and brain region as main factors. Fisher's protected least significant difference test (Fisher's PLSD-test) was used to analyze the statistical significance between groups. The paired Student's t test was used in experiments comparing two groups. In each experiment, a level of p<0.05 was accepted as evidence for a statistically significant effect.

6.1.2 Results In order to determine whether the enhancement of DA and/or NE limited the inhibitory effects associated with (1R,4S)-transnorsertraline on the firing rate of 5-HT neurons, its electrophysiological effects in the DR were evaluated in rats pre-treated with the 5-HT_{1A} receptor antagonist WAY-100635. A one-way ANOVA for the percent of basal firing rate for DR 5-HT neurons indicated an overall significant excitatory effect of treatment factor [F(3,20)=8.2, p<0.001].

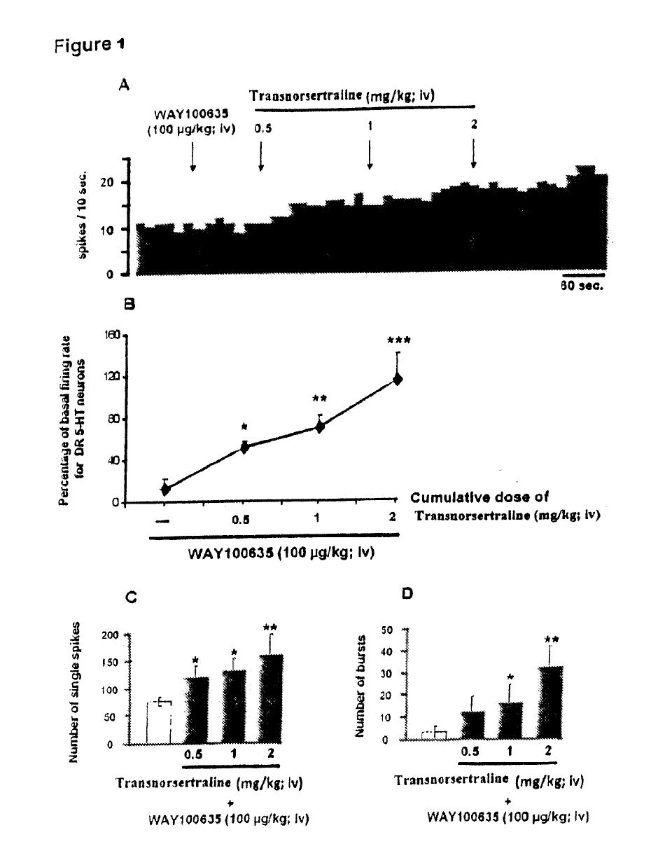

In rats pre-treated with WAY-100635, (1R,4S)-transnorsertraline (0.5-2 mg/kg; iv) elicited a significant increase in DR 5-HT firing rate (FIGS. 1A and 1B). To address the possibility that the excitatory effect of (1R,4S)-transnorsertraline was due to an alteration of single spike and/or burst activity, a more detailed analysis was performed. Two separate one-way ANOVA indicated an overall significant effect of treatment factor for the number of single spikes [F(3,20)=4.5, p<001] and the number of bursts [F(3,20)=3.1, p<0.05]. Thus in rats pre-treated with WAY-100635, (1R,4S)-transnorsertraline significantly increased the number of single spikes and bursts (FIGS. 1C and 1D). In agreement with previous findings (Haddjeri et al., Neuropsychopharmacology 29(10):1800-1806, (2004)), WAY-100635 (100 μg/kg; iv) alone did not alter the spontaneous firing rate of DR 5-HT neurons (1.3±0.1 vs 1.4±0.1 (n=8). Supporting the excitatory effects of (1R,4S)-transnorsertraline on DR 5-HT neurons in conditions of 5-HT_{1A} autoreceptor blockade, there was a significant increase in the number of neurons recorded per track and their mean firing rate after administration of (1R,4S)-transnorsertraline (Table 1). It was previously reported in drug naive rats, a sub-population of DR 5-HT neurons discharged in single-spike mode and bursting activity with two (doublets) or occasionally three spikes (Hajos et al., Neuroscience 69(1):189-187 (1995)). In the present study, about 15% of 5-HT neurons (6/38) displayed a bursting activity. This percentage was doubled by the combination of WAY-100635 and (1R,4S)-transnorsertraline (12/36 neurons), as shown in Table 1 below:

TABLE 1

| | DR 5-HT neurons | |
|---|---|---|
| | Before WAY-100635 + | After WAY-100635 + |
| | (1R,4S)-Transnorsertraline | (1R,4S)-Transnorsertraline |
| Neurons per | 6.3 ± 1.1 | 11.6 ± 1.2* |
| tracks | (n = 38) | (n = 36) |
| Firing rate (Hz) | 1.2 ± 0.1 | 1.8 ± 0.1*** |
| Neurons | 1 ± 0.2 | 4 ± 0.5*** |
| exhibiting | (n = 6) | (n = 8) |
| bursting | | |
| activity | | |
| per track | | |

Consequently, when (1R,4S)-transnorsertraline was acutely administered to rats following an acute intravenous administration of WAY-100635, the discharge of 5-HT neurons was markedly increased. In addition, the mean number of 5-HT neurons recorded per track and their firing rate was significantly enhanced after the combination of WAY-100635 with (1R,4S)-transnorsertraline, therefore suggesting that a subpopulation of 5-HT neurons can be activated in these pharmacological conditions. Moreover, the excitatory effect of (1R,4S)-transnorsertraline, unveiled in the presence of WAY-100635, was characterized by an increase in the number of 5-HT neurons discharging in a doublet spiking activity. Such effects may enhance 5-HT release at nerve terminals to an extent greater than if only single spiking activity had been increased.

In agreement with this, it has been recently demonstrated that the increase in cortical extracellular 5-HT concentrations induced by the concomitant blockade of 5-HT, NE and DA transporters is potentiated by the addition of WAY-100635 to the mix (Weikop et al., Eur. Neuropsychopharmacol. 17(10): 658-671 (2007)). Altogether, these results suggests that the enhancement of noradrenergic and/or dopaminergic transmission exerted a major excitatory role in the regulation of the DR, which limited the inhibitory effect of the SSRI component of (1S,4R)-transnorsertraline on 5-HT neurons, which could be reversed by WAY-100635. Thus, the results suggest that the combination of a transnorsertraline and a selective serotonin 1A receptor agonist/antagonist can provide better efficacy, as well as a faster onset of the effects.

All of the patents, patent applications and publications referred to in this application are incorporated herein in their entireties. Moreover, citation or identification of any reference in this application is not an admission that such reference is available as prior art.

What is claimed is:

1. A method of treating, preventing, or managing a neurological disorder comprising administering to a patient: 1) a therapeutically or prophylactically effective amount of a transnorsertraline, or a pharmaceutically acceptable salt or solvate thereof and 2) a therapeutically or prophylactically effective amount of a serotonin receptor 1A antagonist, or a pharmaceutically acceptable salt, solvate, or stereoisomer thereof.

2. The method of claim 1, wherein the transnorsertraline is (1R,4S)-transnorsertraline.

3. The method of claim 1, wherein the transnorsertraline is (1S,4R)-transnorsertraline.

4. The method of claim 1, wherein the serotonin receptor 1A antagonist is pindolol, WAY-100635, lecozotan, or AV-965.

5. The method of claim 1, wherein the serotonin receptor 1A antagonist is administered prior to the administration of the transnorsertraline.

6. The method of claim 1, wherein the neurological disorder is depression, cognitive deficits, fibromyalgia, pain, a sleep related disorder, chronic fatigue syndrome, attention deficit disorder (ADD), attention deficit hyperactivity disorder (ADHD), restless leg syndrome, schizophrenia, anxiety, obsessive compulsive disorder, posttraumatic stress disorder, seasonal affective disorder (SAD), premenstrual dysphoria, post-menopausal vasomotor symptoms, a neurodegenerative disease, manic conditions, dysthymic disorder, cyclothymic disorder, obesity, or substance abuse or dependency.

7. The method of claim 6, wherein the neurological disorder is depression.

8. A method of treating, preventing, or managing a neurological disorder comprising administering to a patient: 1) a therapeutically or prophylactically effective amount of a transnorsertraline, or a pharmaceutically acceptable salt or solvate thereof; and 2) a therapeutically or prophylactically effective amount of a serotonin receptor 1A agonist, or a pharmaceutically acceptable salt, solvate, or stereoisomer thereof.

9. The method of claim 8, wherein the transnorsertraline is (1R,4S)-transnorsertraline.

10. The method of claim 8, wherein the transnorsertraline is (1S,4R)-transnorsertraline.

11. The method of claim 8, wherein the serotonin receptor 1A agonist is buspirone, eltoprazine, or tandospirone.

12. The method of claim 8, wherein the serotonin receptor 1A agonist is administered prior to the administration of the transnorsertraline.

13. The method of claim 8, wherein the neurological disorder is depression, cognitive deficits, fibromyalgia, pain, a sleep related disorder, chronic fatigue syndrome, attention deficit disorder (ADD), attention deficit hyperactivity disorder (ADHD), restless leg syndrome, schizophrenia, anxiety, obsessive compulsive disorder, posttraumatic stress disorder, seasonal affective disorder (SAD), premenstrual dysphoria, post-menopausal vasomotor symptoms, a neurodegenerative disease, manic conditions, dysthymic disorder, cyclothymic disorder, obesity, or substance abuse or dependency.

14. The method of claim 13, wherein the neurological disorder is depression.

15. A method of treating, preventing, or managing a neurological disorder comprising administering to a patient: 1) a therapeutically or prophylactically effective amount of a transnorsertraline, or a pharmaceutically acceptable salt or solvate thereof; and 2) a therapeutically or prophylactically effective amount of a serotonin receptor 1A modulator, or a pharmaceutically acceptable salt, solvate, or stereoisomer thereof.

16. A pharmaceutical composition comprising: 1) an effective amount of a transnorsertraline, or a pharmaceutically acceptable salt or solvate thereof; 2) an effective amount of a selective serotonin receptor 1A agonist or antagonist, or a pharmaceutically acceptable salt, solvate, or stereoisomer thereof; and 3) a pharmaceutically acceptable carrier or excipient.

17. The pharmaceutical composition of claim 16, wherein the transnorsertraline is (1R,4S)-transnorsertraline.

18. The pharmaceutical composition of claim 16, wherein the transnorsertraline is (1S,4R)-transnorsertraline.

19. The pharmaceutical composition of claim 16, wherein the serotonin receptor 1A antagonist is pindolol, lecozotan, AV-965, or WAY-100635.

20. The pharmaceutical composition of claim 16, wherein the serotonin receptor 1A agonist is tandospirone, buspirone, or eltoprazine.

21. The pharmaceutical composition of claim 16, which further comprises additional active agent.
